(19) United States
(12) Patent Application Publication
(10) Pub. No.: US 2003/0105595 A1
(43) Pub. Date: Jun. 5, 2003

(54) Chi square tests for gene expression

(76) Inventor: Joel S. Bader, Stamford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/196,674

(22) Filed: Jul. 15, 2002

Related U.S. Application Data
(60) Provisional application No. 60/305,482, filed on Jul. 13, 2001.

(51) Int. Cl. G06F 19/00; G01N 33/48; G01N 33/50; G06G 7/48; G06G 7/58

(52) U.S. Cl. 702/19; 703/11

(57) ABSTRACT

The present invention relates to systems and methods that utilize statistical means for analyzing expression of biological samples. Statistical concepts employed include population determinations, normal distributions, correlations between related measures, parameters utilized, Chi Square analysis, degrees of freedom, mean, variance and standard deviations from the mean.

### RELATED APPLICATION

This application claims priority from U.S. Provisional Patent Application Serial No. 60/305,483 filed Jul. 13, 2001, the entirety of which is hereby incorporated by reference.

### FIELD OF THE INVENTION

The present invention relates to systems and methods that utilize statistical means for analyzing expression information of biological samples.

### BACKGROUND OF THE INVENTION

Gene expression varies within populations and even within what appears to be a homogeneous population. The most challenging aspects of presenting gene expression data involve the quantification and qualification of expression values, including standard statistical significance tests and confidence intervals. The current state-of-the-art in array-based studies precludes obtaining standard statistical indices (e.g., confidence intervals, outlier delineation) and performing standard statistical tests (e.g., t-tests, analyses-of-variance) that are used routinely in other scientific domains, because the number of replicates typically present in such studies would ordinarily be considered insufficient for these purposes. Thus, statistical indices and tests are required so estimates can be made about the reliability of observed differences between expression conditions. The key question in these kinds of comparisons is whether it is likely that observed differences in measured values reflect random error only or random error combined with treatment effect (i.e., “true difference”).

### SUMMARY OF THE INVENTION

It is an object of this invention to provide a computer program for gene expression that may be applied to time-series analysis and to drug dose-response studies. It is another object of this invention to use Chi Square analysis to show that variation in gene expression is dependent only upon the average expression level of a gene and not upon the gene per se.

It is yet a further object of this invention to show that Chi Square analysis more clearly distinguishes a low dose from a high dose of a drug when compared to a correlation metric approach that could also be used to analyze identical data.

In one aspect of this invention, expression information of biological samples, as well as their response to external factors is recorded on computer readable media, and is processed by a computer system executing the analysis program of the present invention, or modules thereof. In one aspect, the entry and processing of information is iterative, and a user of the computer system can further manipulate the processing of the expression information. In a preferred embodiment, the processor is capable of producing a model of gene expression patterns based on Chi Square tests, that compares expression of a plurality biological samples in the presence or absence of external factors, and fits a significance value to the model using the Chi Square analysis.

In one aspect, the invention relates to a system for analyzing expression information of biological samples and evaluating statistical variation in expression patterns. In one aspect the biological samples are hybridized on an array. The system involves computer readable media or other such similar reading means that enables the user to cause the system to read expression information of one or more samples from plurality of data sources. The system further involves a processor or similar analyzing means that enables the user to analyze the expression information of the one or more samples, wherein the analysis is performed by estimating variation of expression data. The computer system further involves an interface and selection means that enables the user to select one or more samples presented by the computer system by utilizing the estimated variation in expression patterns from the analyzing means. The system also has a presenting means, such as for example, a CRT or printer that enables the user to cause the system to present the expression information, and the significance of variation of expression of the samples.

In one embodiment, the expression information is obtained from biological samples, which can include a plurality of polynucleotides or polypeptides. In some embodiments, the expression information is obtained from biological samples in the absence of external treatment (X₀) and from biological samples treated or otherwise affected by one or more external factors, such as drugs or environmental conditions. The gene expression information is evaluated in view of a plurality of conditions or levels including high (Xₜ) and low (Yₜ) levels corresponding to time (t). The variation in gene expression is thus estimated by the analyzing means using a Chi Square analysis, wherein the algorithms for such analyses may be read from the computer readable media into the processor or analyzing means, and the results are communicated to the interface, output device or presenting means.

In another embodiment, the computer system calculates the Chi Square value between the samples Xₜ and X₀, which is calculated using standard deviation (SD) according to the relation:

Chi Square=(Xₜ−X₀)²/SD²

In yet another embodiment, the computer system calculates the Chi Square value between the samples Yₜ and X₀, which is calculated using standard deviation (SD) according to the relation:

Chi Square=(Yₜ−X₀)²/SD²

In still another embodiment, the computer system calculates the Chi Square value between the samples Xₜ and Yₜ, which is calculated using standard deviation (SD) according to the relation:

Chi Square=(Xₜ−Yₜ)²/SD²

In one embodiment, the computer system provides a selection means that further enables the user to select the expression of biological samples showing a probability value of false positive equivalent to 1/(total number of biological samples). In one embodiment, the variation is estimated by the processor or analyzing means, and is due to influence of one or more biological samples. In another embodiment, the variation is estimated by the analyzing means, and is due to one or more external factors, (e.g., physical, chemical or biological factors, drugs, genetic or environmental factors) influencing biological samples, or one or more error sources in the process of producing and measuring the samples, including but not limited to, those obtained from hybridization arrays.

In another embodiment, the system is integrated with plurality of processes of array hybridization, image capturing, image analysis, statistical analysis and laboratory information management systems. In another embodiment, the system is integrated with plurality of storage means, such as computer readable media, to store the information from analyzing means. In another embodiment, the selection means and presenting means are a graphical user interface (GUI).

In one embodiment, the system is computer based, and involves a standalone computer. In another embodiment, the system is computer based, and involves one or more networked computers. In this aspect, the analysis can be performed on a plurality of computers in communication with the network.

In still another aspect, the invention provides for methods for analyzing expression information of biological samples, such as array hybridized samples. The method involves the steps of reading expression information of one or more samples from plurality of data sources; analyzing expression information of one or more samples, wherein the analysis is performed by estimating statistical variation; selecting one or more samples by utilizing estimated variation from the analysis; and presenting expression information of one or more biological samples.

In yet another aspect, the invention relates to a computer system for analyzing expression information of array hybridized biological samples, the system including computer readable code (also known as an instruction set) including an algorithm capable of instructing the system processor, reading modules to read expression information of one or more biological samples from a plurality of data sources; analyzing modules to analyze the expression information of the one or more samples, wherein the analysis is performed by estimating stastistical variation; selection modules to select for the one or more analyzed samples by estimating variation from the analyzing modules; and presenting modules to cause the system to present to a user, the expression information of the one or more selected biological samples.

In another aspect, the invention relates to a system including an electronic storage medium having an algorithm, or computer readable code disposed on a data storage means, which allows a user to analyze expression information of biological samples. In this aspect, the storage medium involves computer readable code having an algorithm that enables a processor to allow a user to read expression information of one or more biological samples from a plurality of data sources; computer readable code that enables a processor to allow a user to analyze the expression information of one or more samples, wherein the analysis is performed by estimating statistical variation; computer readable code that enables a processor to allow a user to select one or more samples by utilizing estimated variation from analyzing means; and computer readable code that enables a processor to allow a user to cause the computer system to present the expression information of one or more samples. In one embodiment, the algorithm has one or more modules or subroutines. In another embodiment, the computer system includes a reading means capable of reading physically measured information from a plurality of array locations having biological samples, i.e., a plurality of replications of the samples. In yet another embodiment, the computer system includes an array of biological samples, wherein the biological samples are hybridized to their reverse complement sequences or fragments thereof, and the hybridization is carried out on a glass substrate.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 illustrates standard deviation in band intensity for 13K bands versus the intensity of each band.

FIG. 2 shows bands that exhibited a difference in p-value of <10⁻⁵ in at least 2 of the following 3 comparisons: low dose versus vehicle, high dose versus vehicle, and low dose versus high dose, for at least 2 drugs.

FIG. 3 illustrates band clustering using “k-clustering” means for all 13K bands.

FIG. 4 illustrates band clustering using “k-clustering” means for the 2K bands that meet the significance and filter tests shown in FIG. 2.

FIG. 5 shows Chi Square metric and traditional correlation coefficient interpretations of results from all 13K bands.

FIG. 6 shows a comparison of analytic tests done on only the best 2K bands, and the better distinction between high and low dose levels provided by the Chi Square metric.

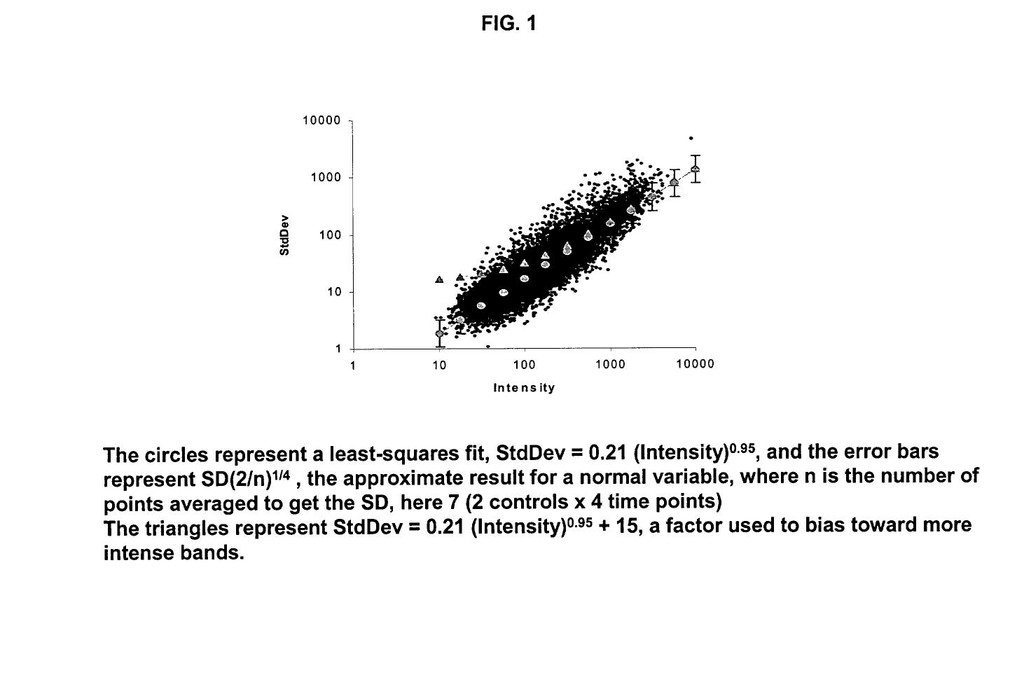

### DETAILED DESCRIPTION OF THE INVENTION

Chi Square analysis is a statistical calculation used in genetics studies. A calculated value of Chi Square compares the frequencies of various kinds of items in a random sample to the expected frequencies of those items if the population from which the items are drawn is what an investigator has hypothesized. That is, Chi Square analysis will tell how well the hypothesized frequency fits with the set of frequencies obtained from a random sample.

Integral to Chi Square analysis is the concept of standard deviation from the mean. Standard deviation is an index of variability that shows dispersion among measures within a given population. Variation in levels of gene expression are held to depend on any number of factors, not the least of which is the gene per se, and its expression according to biologic influences impacting it, such as, for example, drugs.

As used herein, “biological samples” refers to polynucleotides, including polyribonucleotides and polydeoxyribonucleotides, as well as polypeptides. Biological samples include, derivatives of polypeptides and polynucleotides, as well as other varients, for example allelic polymorphisms and single nucleotide polymorphisms (SNP's). According to the present invention, the biological samples can be processed, for example in an array format, such as an array or matrix of immobilized polynucleotides that are probed or hybridized to their reverse complement sequence, or a portion thereof.

As used herein, the term “statistical variation” refers to Chi Square analysis, population determinations, normal distributions, correlations between related measures, parameters utilized, degrees of freedom, mean, variance and standard deviations from the mean.

As used herein, the terms “a computer-based system” or “computer system” are used interchangeably and refer to the hardware means, software means, and data storage means used to analyze the expression levels of genes. The minimum hardware means of the computer-based systems of the present invention include a central processing unit (CPU), input means, output means, and data storage means. Those skilled in the art will readily appreciate that any of the currently available computer-based systems are suitable for use in the present invention. It is understood that any general or special purpose system includes a processor in electrical communication with both a memory and at least one input/output device, such as a terminal. Such a system may include, but is not limited to, personal computers, workstations or mainframes. The processor may be a general purpose processor or microprocessor or a specialized processor executing programs located in RAM memory. The programs may be placed in RAM from a storage device, such as a disk or preprogrammed ROM memory. In one embodiment, the RAM memory is used both for data storage and program execution. The term computer system also embraces systems where the processor and memory reside in different physical entities but which are in electrical communication by means of a network.

As used herein, the term “computer readable media” refers to any data storage medium which can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any computer readable media can be used to create a manufacture comprising computer readable medium having recorded thereon a program of the present invention. The choice of the data storage medium will generally be based on the means chosen to access the stored information.

As used herein, the term “data storage means” refers to memory which can store expression information of the present invention, or a memory access means which can access manufactures having recorded thereon the expression information of the present invention.

As used herein, the term “recorded” refers to a process for storing information on computer readable medium. A skilled artisan can readily adopt any known method for recording information on computer readable medium to generate manufactures involving the program information of the present invention. A variety of data storage structures are available to a skilled artisan for creating a computer readable medium having recorded thereon a computer readable program or algorithm of the present invention. In addition, a variety of data processor programs and formats can be used to store the information of the present invention on computer readable medium.

As used herein the term “external factors” refers to physical, chemical, biological, or environmental factors that modulate the expression or activity of biological samples. “External treatment” refers to the exposure of one or more biological samples to one or more external factors. An example of a chemical external factor is a naturally occuring or synthesized agent, that is not itself a native component of the biological samples, including but not limited to, a drug or toxin. An example of a biological external factor is a agent normally present in a living cell, that is not itself a native component of the biological samples but that acts on the biological samples, for example but not limited to a hormone or a gene product such as one from one or more genes in an upstream metabolic pathway relative to the biological samples, or a gene product of a microorganism (i.e., a bactrium, a virus, a fungus, or microbe). An example of an environmental external factor is a naturally occuring or manufactured agent or stimulus, that is not itself a native component of the biological samples, for example but not limited to radiation such as UV, electromagnetic energy, or the like. An example of a phycical external factor is heat, cold, or pressure. One skilled in the art will recognize, for example, that an external factor may be both a chemical and biological factor, or an environmental and chemical factor, or an environmental and biological factor, or all three.

The output of the computer system can be represented in a word processing text file, formatted in commercially-available software such as WordPerfect® and Microsoft Word®, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the expression information of the present invention.

Chi Square analysis provides a system and method for determining statistical variation in gene expression levels that depends upon the average expression level of the gene and not upon the gene per se. That is, two genes with the same mean expression level exhibit the same statistical variation from sample to sample, which can be detected and interpreted by the system of the present invention.

The determination of expression levels for polynucleotides and polypeptides are well known in the art. For example, RT-PCR provides a quantitative method for determining expression of one or more genes. The quantitative expression of various biological samples is assessed using, for example microtiter plates containing RNA samples from a variety of normal and pathology-derived cells, cell lines and tissues using real time quantitative PCR (RTQ PCR). RTQ PCR is performed, e.g., an Applied Biosystems ABI PRISM® 7700 or an ABI PRISM® 7900 HT Sequence Detection System. The capabilities of RT-PCR in determining gene expression information from biological samples is well known to those trained in the field. RT-PCR is accomplished using primers of standard designs to skilled artisans.

Southern or Northern blotting provides another method of detecting the presence and the expression of one or more genes, and such hybridizations can additionally be perfomed on arrays or in matrix format, e.g. dot blots or hybridization on glass arrays as part of a high throughput screen. These techniqies are well described in the scientific literature. Probes useful to these techniques are reverse complements of the target sequences, including fragments of these greater than 20 nucleotides in length. The creation and use of such probes are well known to skilled artisans.

Alternatively, fluorescent activated cell sorting (FACS) or Western blotting provide two non-limiting examples of methods for determining expression levels of polypeptides. One example of the use of fluorescence detection for cancer screening is U.S. Pat. No. 5,270,171 to Cercek, et al, which teaches a method to identify, separate and purify the factor or factors that provoke a response by SCM (structuredness of the cytoplasmic matrix) responding lymphocytes. Also, U.S. Pat. No. 5,554,505 to Hajek, et al. discloses an optical screening method and apparatus for identifying both the morphology and selective characteristics or properties expressed by cells. Similarly, U.S. Pat. No. 5,562,114 to Chu, et al. discloses a diagnostic immunoassay method using monoclonal antibodies. Each of the above references are hereby incorporated hirein in their entireties.

Depending upon the size of the biological sample, the collected sample within the specimen could be processed, for example on a standard flow cytometer even prior to initial observation under the fluorescence microscope. In the case of large volumes of fluid contained within a biological sample, such as one would obtain on a screen for uterine cervix or colon cancer, a flow cytometer can process large volumes of cells in a very short period of time. Commercially available flow cytometers may be used. Manufacturers of such devices include Coulter, Becton-Dickenson, and Cytomation. As well understood in the art, flow cytometry involves the suspension of individual cells in a solution, followed by the movement of the cells through a tubular system which only allows one cell at a time to flow. The cells pass through a chamber in the system where there is a selection of lasers of selected different frequencies of light to conduct a number of measurements, including cell counts, cell measurements as to overall size and other parameters.

As applied to the method of this invention, the flow cytometer would have a selection of lasers which provide light to match the excitation frequency of the particular compound used to produce fluorescence in the cell sample. By this fluorescent tagging, the flow cytometer is able to accurately count the number of cells which fluoresce. In order to actually separate fluorescing versus non-fluorescing cells, the FACS must be conducted. The FACS involves the inducement of a charge (positive or negative) on the cell surface of each cell which passes through the flow cytometer. By this induced charge, the fluorescing cells are separated from non-fluorescing cells. Accordingly, the fluorescing cells would be placed into a separate container from the non-fluorescing cells. As discussed above, this concentrated sample of fluorescing cells makes viewing easier under a fluorescence microscope, and for easier photographing of the fluorescing cells. Often, it is desirable first to attempt to locate fluorescing cells by simply viewing them under a fluorescence microscope. The capabilities of flow cytometers and fluorescence activated cell sorters are well known to those trained in the field. However, it shall be understood that the invention is not limited to any particular sequence in terms of the use of the present flow cytometer, fluorescence microscope, or by determining expression by conducting a FACS analysis. Instead the present invention is concerned with obtaining a biological sample and assigning an initial estimate as to the expression of one or more genes from that sample, to determine the data fit of a meaningful hypothesis by Chi Square analysis.

To practice the invention, one skilled in the art obtains a source of biological samples, as well as expression information about the biological samples, i.e., expression information relative to one or more genes or polypeptides encoded therein. Data is collected for a particular phenotype appearing within a given population under study, whereby expression of that phenotype is to be determined within the biological sample under study. The phenotype is correlated to a specific genotype, which is then represented as a band with a given intentity, thus indicative of gene expression levels, and is then compared to control samples. Alternatively, the response of a gene to dosage with one or more drugs is determined by band intensity. The standard deviation from the mean (StdDev) is calculated for the control samples, and is fit to functional form in the following equation:

log(StdDev)=A+B log(intensity)

The Chi Square is then calculated for the significance of the gene under study, for example but not limited to, low or high dose of drug versus control, using the equation:

Chi Square=sum of (x(t)−x0)²/SD²

where x(t) is the dose of drug at time, t; x0 is the mean of the control values over all times; df is the number of times sampled; and p-value is the tail of the Chi Square distribution. When values are provided and calculations performed, it can be determined if there are significant differences between expression patterns, for example, gene expression in view of high and low drug doses versus control values.

To determine further if there is significance between high drug dose versus low drug dose, the following calculation is performed:

Chi Square=sum of (x(t)−y(t))²/SD²

where x(t) is the high dose of drug at time, t; y(t) is the low dose of drug at time, t; and df is 2×the number of times given.

In order to achieve a low, false-positive rate, a gene or feature is selected that shows “significance” at a p-value=1/(total number of genes or features). Ideally, it is required is that the same gene or feature shows significance for tests involving a minimum of 2 drugs, and for each drug tested, 2 of the following 3 parameters are triggered: low dose versus vehicle, high dose versus vehicle and low dose versus high dose.

With respect to filters used, the equation:

StdDev=A(Intensity)^{B}+an allowance factor

provides a bias to high intensity. To avoid misrepresentations in data due to polymorphisms, it is important that the Chi Square value be significant. This can be achieved by ignoring the largest difference in calculating the Chi Square value. An appropriate filter is then selected by choosing the ratio of larger difference for: where 1 log(ratio) 1>log (2) provides a reasonable cut-off.

These equations are stored in computer readable media as part of a computer readable program written in a suitable language, for example C, C++, UNIX, FORTRAN, BASIC, PASCAL, or the like. The program provides an algorithm for performing the Chi Square analysis, as well as other functional elements contained in one or more modules or subroutines (e.g., relational database capabilities, search features, and user defined functions). An example of such an algorithm is provided as Example 1. The algorithm includes reading modules that enable the user to permit the system to read the expression information of one or more biological samples from plurality of data sources input by the user or by automated means; analyzing modules that enable the user to analyze the expression information of one or more biological samples, wherein the analysis is performed by estimating sample variation; selection modules that enable the user to select one or more samples by utilizing the estimated statistical variation from the analyzing modules; and presenting modules that enable the user to cause the system to present the expression information of one or more samples.

For example, data on gene expression, drug type and dosages, and the like is entered by a user into the computer system, (see, Example 2) where it is stored in memory and manipulated by the processor using the modules of the algorithm described. The output information is thus available to the user, and is visualized by an output device such as a graphical user interface, or a printed copy. The output information permits a determination of significance of a comparison of at least two biological samples.

### EXAMPLE 1

### Computer Algorithm for Determining Expression Variance

The following provides an implementation of the factorial experimental design of this invention as a computer program in the C language. This code can run on any computers recognizing this language, for example but not limited to PowerPC™ based Macintosh type computers and Pentium™ based PC type computers.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

### EXAMPLE 2

### Determining Expression Variance

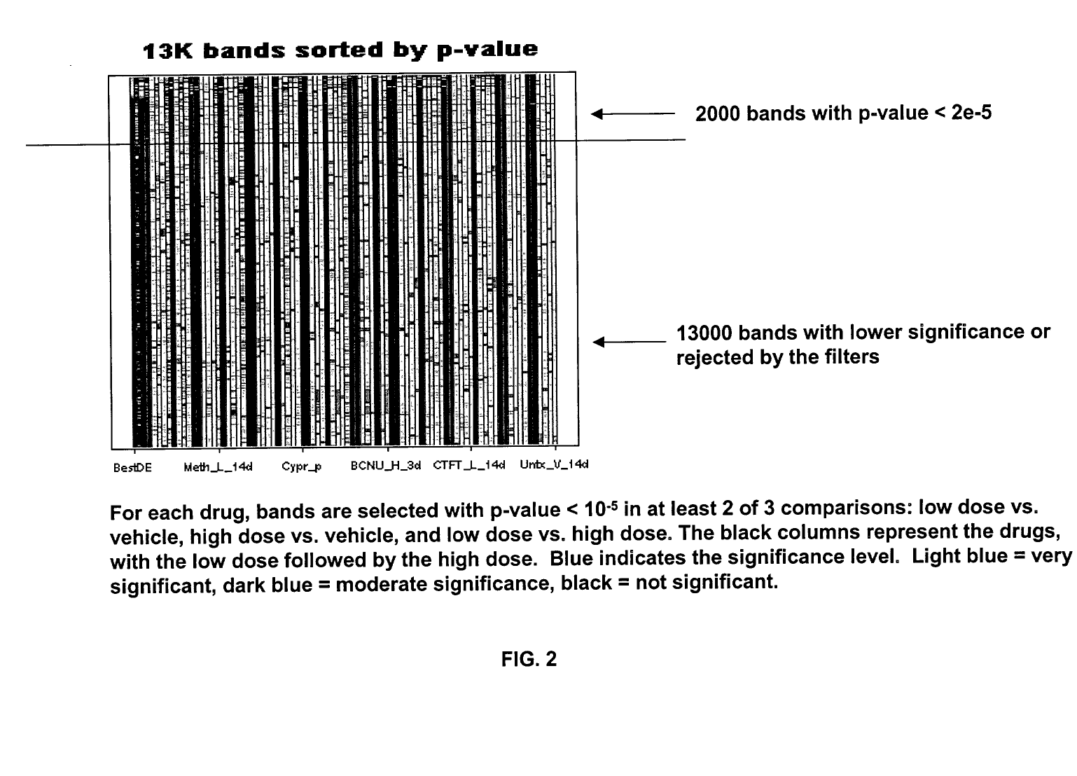

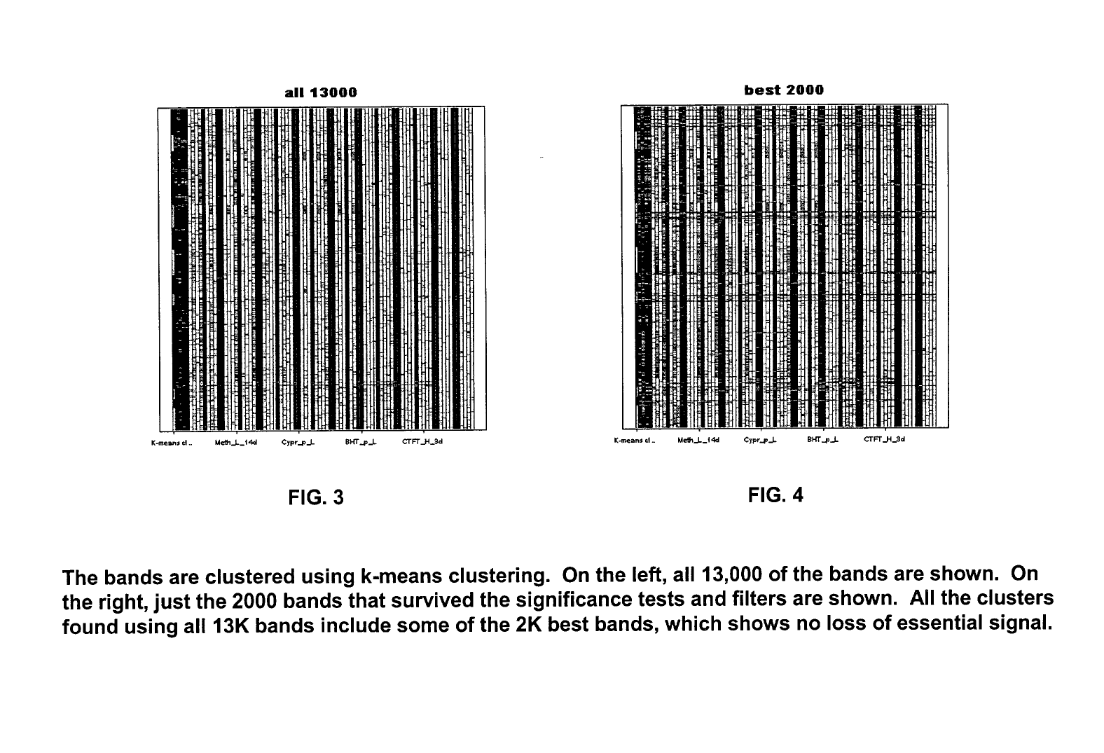

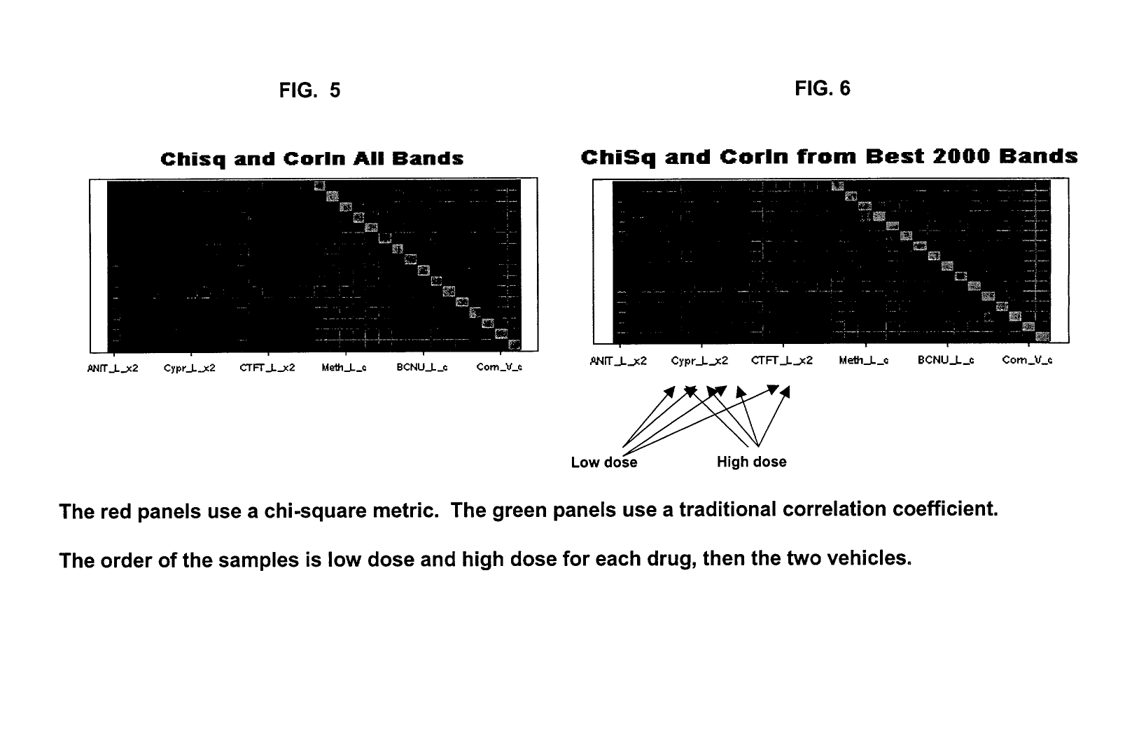

A multi-sample expression analysis was run using the program described in Example 1. For each drug run, only runs for genes producing bands with a p-value<10⁻⁵ in at least 2 of the following 3 comparisons were selected: low dose versus vehicle, high dose versus vehicle and low dose versus high dose. Bands that exhibited this difference for at least 2 drugs were chosen (FIG. 2). Since clusters found using all 13K bands included some of the best 2K bands, it was concluded that no essential signal was lost (see FIGS. 3 and 4). As seen in FIG. 5, Chi Square metric and traditional correlation coefficient interpretations of the results from all 13K bands. A comparison using the same analytic tests on only the best 2K bands. It can be seen that the Chi Square metric provides a better distinction between low and high doses than the correlation metric, which is not able to distinguish between the low dose and the high dose (see FIG. 6). The low-dose/high dose statistical variation is more pronounced when only the best 2K bands are selected.

DATA INPUT

nDim 3
Drug 9
ANIT
Methylenedianiline
Dichlorobenzene
Cyproterone
BCNU
BHT
CTFT
Corn Oil
Untx
Dose 3
Vehicle
Low
High
Time 4
1d
3d
7d
14d
nSample 59

| | | | | |
|---|---|---|---|---|
| 0 | 15825 | ANIT | High | 1d |
| 1 | 15842 | ANIT | High | 3d |
| 2 | 15859 | ANIT | High | 7d |
| 3 | 15873 | ANIT | Low | 14d |
| 4 | 15826 | ANIT | Low | 1d |
| 5 | 15841 | ANIT | Low | 3d |
| 6 | 15858 | ANIT | Low | 7d |
| 7 | 15834 | BCNU | High | 1d |
| 8 | 15850 | BCNU | High | 3d |
| 9 | 15833 | BCNU | Low | 1d |
| 10 | 15849 | BCNU | Low | 3d |
| 11 | 15866 | BCNU | Low | 7d |
| 12 | 15880 | BHT | High | 14d |
| 13 | 15832 | BHT | High | 1d |
| 14 | 15848 | BHT | High | 3d |
| 15 | 15865 | BHT | High | 7d |
| 16 | 15878 | BHT | Low | 14d |
| 17 | 15831 | BHT | Low | 1d |
| 18 | 15847 | BHT | Low | 3d |
| 19 | 15864 | BHT | Low | 7d |
| 20 | 15877 | CTFT | High | 14d |
| 21 | 15830 | CTFT | High | 1d |
| 22 | 15846 | CTFT | High | 3d |
| 23 | 15863 | CTFT | High | 7d |
| 24 | 15876 | CTFT | Low | 14d |
| 25 | 15829 | CTFT | Low | 1d |
| 26 | 15845 | CTFT | Low | 3d |
| 27 | 15862 | CTFT | Low | 7d |
| 28 | 15884 | Corn Oil | Vehicle | 14d |
| 29 | 15838 | Corn Oil | Vehicle | 1d |
| 30 | 15855 | Corn Oil | Vehicle | 3d |
| 31 | 15870 | Corn Oil | Vehicle | 7d |
| 32 | 15882 | Cyproterone | High | 14d |
| 33 | 15836 | Cyproterone | High | 1d |
| 34 | 15853 | Cyproterone | High | 3d |
| 35 | 15868 | Cyproterone | High | 7d |
| 36 | 15835 | Cyproterone | Low | 1d |
| 37 | 15852 | Cyproterone | Low | 3d |
| 38 | 15867 | Cyproterone | Low | 7d |
| 39 | 15872 | Dichlorobenzene | High | 14d |
| 40 | 15824 | Dichlorobenzene | High | 1d |
| 41 | 15840 | Dichlorobenzene | High | 3d |
| 42 | 15857 | Dichlorobenzene | High | 7d |
| 43 | 15871 | Dichlorobenzene | Low | 14d |
| 44 | 15823 | Dichlorobenzene | Low | 1d |
| 45 | 15839 | Dichlorobenzene | Low | 3d |
| 46 | 15856 | Dichlorobenzene | Low | 7d |
| 47 | 15875 | Methylenedianiline | High | 14d |
| 48 | 15828 | Methylenedianiline | High | 1d |
| 49 | 15844 | Methylenedianiline | High | 3d |
| 50 | 15861 | Methylenedianiline | High | 7d |
| 51 | 15874 | Methylenedianiline | Low | 14d |
| 52 | 16303 | Methylenedianiline | Low | 1d |
| 53 | 15843 | Methylenedianiline | Low | 3d |
| 54 | 15860 | Methylenedianiline | Low | 7d |
| 55 | 15883 | Untx | Vehicle | 14d |
| 56 | 15837 | Untx | Vehicle | 1d |
| 57 | 15854 | Untx | Vehicle | 3d |
| 58 | 15869 | Untx | Vehicle | 7d |

DATA OUTPUT

| | |
|---|---|
| 12955 | F Pvalue 100670_Pooled_ANIT_-_High_-_1d |
| Band | |

| | 100686_Pooled_ANIT_-_High_-_3d | | | | |
|---|---|---|---|---|---|
| | 100702_Pooled_ANIT_-_High_-_7d | | | | |
| | 100716_Pooled_ANIT_-_Low_-_14d | | | | |
| | 100669_Pooled_ANIT_-_Low_-_1d | | | | |
| | 100685_Pooled_ANIT_-_Low_-_3d | | | | |
| | 100701_Pooled_ANIT_-_Low_-_7d | | | | |
| | 100678_Pooled_BCNU_-_High_-_1d | | | | |
| | 100694_Pooled_BCNU_-_High_-_3d | | | | |
| | 100677_Pooled_BCNU_-_Low_-_1d | | | | |
| | 100693_Pooled_BCNU_-_Low_-_3d | | | | |
| | 100709_Pooled_BCNU_-_Low_-_7d | | | | |
| | 100722_Pooled_BHT_-_High_-_14d | | | | |
| | 100676_Pooled_BHT_-_High_-_1d | | | | |
| | 100692_Pooled_BHT_-_High_-_3d | | | | |
| | 100708_Pooled_BHT_-_High_-_7d | | | | |
| | 100721_Pooled_BHT_-_Low_-_14d | | | | |
| | 100675_Pooled_BHT_-_Low_-_1d | | | | |
| | 100691_Pooled_BHT_-_Low_-_3d | | | | |
| | 100707_Pooled_BHT_-_Low_-_7d | | | | |
| | 100720_Pooled_CTFT_-_High_-_14d | | | | |
| | 100674_Pooled_CTFT_-_High_-_1d | | | | |
| | 100690_Pooled_CTFT_-_High_-_3d | | | | |
| | 100706_Pooled_CTFT_-_High_-_7d | | | | |
| | 100719_Pooled_CTFT_-_Low_-_14d | | | | |
| | 100673_Pooled_CTFT_-_Low_-_1d | | | | |
| | 100689_Pooled_CTFT_-_Low_-_3d | | | | |
| | 100705_Pooled_CTFT_-_Low_-_7d | | | | |
| | 100727_Pooled_Corn_Oil_Vehicle_-_14d | | | | |
| | 100682_Pooled_Corn_Oil_Vehicle_-_1d | | | | |
| | 100698_Pooled_Corn_Oil_Vehicle_-_3d | | | | |
| | 100713_Pooled_Corn_Oil_Vehicle_-_7d | | | | |
| | 100725_Pooled_Cyproterone_-_High_-_14d | | | | |
| | 100680_Pooled_Cyproterone_-_High_-_1d | | | | |
| | 100696_Pooled_Cyproterone_-_High_-_3d | | | | |
| | 100711_Pooled_Cyproterone_-_High_-_7d | | | | |
| | 100679_Pooled_Cyproterone_-_Low_-_1d | | | | |
| | 100695_Pooled_Cyproterone_-_Low_-_3d | | | | |
| | 100710_Pooled_Cyproterone_-_Low_-_7d | | | | |
| | 100715_Pooled_Dichlorobenzene_-_High_-_14d | | | | |
| | 100668_Pooled_Dichlorobenzene_-_High_-_1d | | | | |
| | 100684_Pooled_Dichlorobenzene_-_High_-_3d | | | | |
| | 100700_Pooled_Dichlorobenzene_-_High_-_7d | | | | |
| | 100714_Pooled_Dichlorobenzene_-_Low_-_14d | | | | |
| | 100667_Pooled_Dichlorobenzene_-_Low_-_1d | | | | |
| | 100683_Pooled_Dichlorobenzene_-_Low_-_3d | | | | |
| | 100699_Pooled_Dichlorobenzene_-_Low_-_7d | | | | |
| | 100718_Pooled_Methylenedianiline_-_High_-_14d | | | | |
| | 100672_Pooled_Methylenedianiline_-_High_-_1d | | | | |
| | 100688_Pooled_Methylenedianiline_-_High_-_3d | | | | |
| | 100704_Pooled_Methylenedianiline_-_High_-_7d | | | | |
| | 100717_Pooled_Methylenedianiline_-_Low_-_14d | | | | |
| | 100671_Pooled_Methylenedianiline_-_Low_-_1d | | | | |
| | 100687_Pooled_Methylenedianiline_-_Low_-_3d | | | | |
| | 100703_Pooled_Methylenedianiline_-_Low_-_7d | | | | |
| | 100726_Pooled_Untx_-_Series_1_-_14d | | | | |
| | 100681_Pooled_Untx_-_Series_1_-_1d | | | | |
| | 100697_Pooled_Untx_-_Series_1_-_3d | | | | |
| | 100712_Pooled_Untx_-_Series_1_-_7d | | | | |
| b1i0_51.7 | 0 | 23.544892 | 30.103037 | 21.567616 | 81.997346 |
| | 30.702796 | 22.373736 | 20.666184 | 29.608189 | 30.974794 |
| | 24.268556 | 21.637515 | 19.759957 | 26.784709 | 24.213618 |
| | 25.0702 | 17.715696 | 18.314255 | 18.782205 | 18.023983 |
| | 12.127385 | 19.058594 | 17.270417 | 23.529339 | 13.918661 |
| | 32.701469 | 22.809197 | 30.635824 | 17.663446 | 15.672605 |
| | 21.538437 | 14.610615 | 20.769767 | 19.899767 | 35.069505 |
| | 32.34381 | 22.634416 | 19.269776 | 21.873214 | 12.769259 |
| | 21.769265 | 27.835384 | 10.749258 | 16.630761 | 23.430812 |
| | 28.93028 | 26.061803 | 16.565025 | 33.183899 | 23.54918 |
| | 27.879463 | 23.351387 | 71.331804 | 22.283294 | 27.013604 |
| | 21.369566 | 14.763453 | 27.838359 | 17.47643 | 18.055182 |
| b1i0_53.0 | 0 | 99.498789 | 84.157358 | 106.268871 | 54.672107 |
| | 121.830758 | 45.024387 | 108.124859 | 110.830028 | 118.438436 |
| | 117.109022 | 98.959691 | 104.154649 | 82.870158 | 104.264211 |
| | 101.737289 | 104.082898 | 96.381843 | 108.777447 | 87.177289 |
| | 104.467173 | 76.60183 | 96.215713 | 82.040827 | 93.627534 |
| | 91.49095 | 116.854083 | 111.788057 | 113.540644 | 103.105961 |
| | 79.614576 | 97.690274 | 114.849913 | 40.970251 | 93.392231 |
| | 99.723751 | 99.01235 | 111.604202 | 99.850245 | 99.008207 |
| | 95.421533 | 103.413865 | 48.520724 | 99.659889 | 116.359037 |
| | 127.083002 | 71.532666 | 113.83161 | 60.924121 | 101.131133 |
| | 98.113818 | 113.819446 | 55.869889 | 103.357971 | 101.135801 |
| | 111.54306 | 104.211595 | 88.659637 | 102.327053 | 100.549388 |
| b1i0_54.5 | 0 | 102.162121 | 114.236918 | 95.668457 | 33.492072 |
| | 105.289908 | 111.329988 | 88.171497 | 112.394852 | 112.08911 |
| | 102.765858 | 103.605607 | 108.188735 | 93.418658 | 82.822941 |
| | 96.924635 | 81.737892 | 91.728547 | 93.969467 | 101.517293 |
| | 92.421443 | 79.055276 | 92.290305 | 88.546772 | 79.535788 |
| | 114.276011 | 107.896665 | 98.390101 | 102.227042 | 120.852053 |
| | 95.560027 | 89.0957 | 91.858273 | 69.777006 | 98.482402 |
| | 80.834446 | 81.205749 | 94.406498 | 93.730905 | 98.196724 |
| | 84.90956 | 91.994075 | 89.822644 | 80.917888 | 101.411635 |
| | 88.394326 | 128.360502 | 88.11377 | 51.093146 | 104.866521 |
| | 105.968709 | 82.178127 | 62.552039 | 123.714173 | 111.42007 |
| | 86.490276 | 112.252632 | 111.766402 | 94.787428 | 100.08862 |
| b1i0_57.4 | 0 | 1272.181176 | 712.350351 | 685.827476 | 810.740232 |
| | 1209.0048 | 1266.179729 | 1302.501456 | 1153.585685 | 1296.093934 |
| | 1234.82714 | 1366.747949 | 1344.875534 | 1048.923128 | 1176.845905 |
| | 1234.732576 | 990.188001 | 1228.87043 | 1181.508899 | 1224.46485 |
| | 1156.307983 | 1118.740972 | 1069.945925 | 1131.011927 | 1134.759409 |
| | 1314.63168 | 1320.211382 | 1266.951876 | 1269.838966 | 1421.83629 |
| | 1212.104564 | 1374.303669 | 1609.013097 | 775.476752 | 1104.833598 |
| | 1020.813029 | 1104.711819 | 1285.38766 | 1337.738793 | 1436.846037 |
| | 1142.184413 | 1259.846692 | 1064.023452 | 1287.17738 | 1338.104131 |
| | 1300.991332 | 1352.070182 | 1297.569152 | 455.917146 | 1229.664203 |
| | 1406.835021 | 1183.591626 | 789.213457 | 1376.007231 | 1432.65909 |
| | 1280.075113 | 1124.261263 | 1188.88269 | 1169.56077 | 1431.349855 |
| b1i0_60.7 | 0 | 74.318131 | 74.885892 | 72.634322 | 141.739709 |
| | 87.084708 | 67.382771 | 75.420732 | 78.554664 | 115.43014 |
| | 88.974793 | 80.086747 | 96.794687 | 68.414051 | 71.521922 |
| | 70.086835 | 62.308954 | 90.107901 | 72.362149 | 69.126883 |
| | 79.01462 | 66.335668 | 47.716031 | 61.290957 | 61.305902 |
| | 95.315202 | 80.425505 | 71.714776 | 83.221031 | 73.39388 |
| | 73.23158 | 80.209404 | 98.748627 | 57.479996 | 53.895513 |
| | 46.420581 | 73.60713 | 76.154166 | 72.42433 | 80.221557 |
| | 82.423227 | 79.936554 | 62.132235 | 77.865835 | 94.457296 |
| | 95.980356 | 58.606575 | 74.814424 | 158.258085 | 67.134602 |
| | 85.720582 | 82.440553 | 139.591736 | 66.657168 | 78.988282 |
| | 79.323633 | 66.614629 | 85.62878 | 79.061055 | 84.584349 |
| b1i0_62.5 | 0 | 49.333039 | 93.55836 | 81.396541 | 249.285581 |
| | 68.444181 | 104.914862 | 91.612585 | 67.749485 | 84.846006 |
| | 69.477608 | 57.902516 | 82.577232 | 57.739134 | 81.055744 |
| | 56.242769 | 81.593016 | 63.696139 | 74.661691 | 70.37307 |
| | 62.541429 | 41.230387 | 65.175429 | 55.656819 | 77.005689 |
| | 66.074812 | 64.308598 | 67.003095 | 83.061617 | 64.193046 |
| | 54.851502 | 84.924007 | 72.738439 | 148.303315 | 49.192872 |
| | 64.931492 | 71.818063 | 62.408898 | 59.374251 | 70.853701 |
| | 64.49277 | 68.450513 | 97.82131 | 76.856718 | 68.637928 |
| | 73.54091 | 76.310018 | 88.914069 | 185.668673 | 59.418237 |
| | 70.638693 | 71.551375 | 207.597461 | 63.428999 | 69.922381 |
| | 86.010994 | 51.796498 | 65.940154 | 79.383289 | 76.349883 |
| b1i0_63.7 | 0 | 50.086148 | 0.013986 | 147.855112 | 9.84461 |
| | 170.563405 | 7.244774 | 299.354374 | 98.176856 | 115.095918 |
| | 155.387462 | 104.211339 | 204.187792 | 41.881109 | 240.682376 |
| | 120.102063 | 313.443844 | 161.614678 | 152.614587 | 171.352528 |
| | 132.26082 | 70.808399 | 91.065597 | 90.26942 | 203.400877 |
| | 78.723363 | 113.76279 | 153.65371 | 214.990675 | 77.290828 |
| | 67.22041 | 215.288026 | 120.071582 | 18.516288 | 51.361368 |
| | 111.761376 | 154.734763 | 68.203119 | 110.350994 | 146.033635 |
| | 157.667328 | 128.994118 | 5.697943 | 224.273133 | 152.140783 |
| | 166.160213 | 24.943422 | 258.765787 | 2.07316 | 85.097835 |
| | 153.773953 | 144.320995 | 0.352662 | 24.89242 | 84.097621 |
| | 287.243053 | 32.43036 | 60.797084 | 160.349147 | 186.394024 |
| b1i0_69.4 | 0 | 208.615745 | 284.116754 | 271.180949 | 748.935515 |
| | 276.30951 | 370.530811 | 305.405868 | 194.274676 | 219.097168 |
| | 254.034511 | 292.140705 | 325.132969 | 268.812266 | 241.720276 |
| | 251.241121 | 255.877557 | 286.544691 | 272.460758 | 190.569808 |
| | 282.903132 | 200.797981 | 70.194874 | 266.958137 | 223.117159 |
| | 388.812807 | 274.075638 | 351.831761 | 323.241725 | 356.574433 |
| | 106.637994 | 308.845886 | 219.856445 | 528.340834 | 254.857169 |
| | 250.680914 | 317.87817 | 243.165372 | 270.664752 | 326.261078 |
| | 311.303114 | 295.499652 | 393.194329 | 261.660532 | 333.2913 |
| | 130.611455 | 334.562355 | 304.302031 | 512.877262 | 262.228392 |
| | 329.88134 | 290.2061 | 714.823366 | 349.369569 | 349.278273 |
| | 303.138007 | 298.088233 | 242.026718 | 325.630603 | 293.444945 |

### EQUIVALENTS

From the foregoing detailed description of the specific embodiments of the invention, it should be apparent that a unique procedure to evaluate time-series and drug-dose responses in gene expression studies has been described. Although particular embodiments have been disclosed herein in detail, this has been done by way of example for purposes of illustration only, and is not intended to be limiting with respect to the scope of the appended claims that follows. In particular, it is contemplated by the inventor that substitutions, alterations, and modifications may be made to the invention without departing from the spirit and scope of the invention as defined by the claims.

1. A computer system for analyzing expression information of biological samples, the system comprising:
reading means to read expression information of one or more samples from plurality of data sources;
analyzing means to analyze the read expression information of the one or more samples, wherein the analysis is performed by estimating a statistical variation;
selection means to select one or more analyzed biological samples by utilizing estimated variation from the analyzing means; and
presenting means to cause the system to present the expression information of one or more samples.

2. The computer system of claim 1, wherein the expression information is obtained from biological samples having no external treatment (X₀) and biological samples treated by one or more external factors at plurality of levels including high (Xₜ) and low (Yₜ) levels corresponding to time (t), and wherein the statistical variation is estimated by analyzing means using a Chi Square analysis.

3. The computer system according to claim 2, wherein a Chi Square value is calculated using standard deviation (SD) according to the relation
Chi Square=(Xₜ−X₀)²/SD².

4. The computer system according to claim 2, wherein a Chi Square value is calculated using standard deviation (SD) according to the relation
Chi Square=(Yₜ−X₀)²/SD².

5. The computer system according to claim 2, wherein a Chi Square value is calculated using standard deviation (SD) according to the relation
Chi Square=(Xₜ−Yₜ)²/SD².

6. The computer system according to claim 1, wherein the selection means further selects the expression of biological samples showing a probability value of false positive equivalent to
1/(total number of biological samples).

7. The computer system according to claim 1, wherein the variation estimated by the analyzing means is due to influence of one or more external factors.

8. The computer system according to claim 1, wherein the variation estimated by the analyzing means is due to one or more biological factors influencing biological samples.

9. The computer system according to claim 1, wherein the variation estimated by the analyzing means is due to one or more error sources in the process of producing and measuring hybridized arrays.

10. The computer system according to claim 1, wherein the biological samples are a plurality of polynucleotides.

11. The computer system according to claim 1, wherein the biological samples are a plurality of polypeptides.

12. The computer system according to claim 8, wherein the external factor is a drug.

13. The computer system according to claim 1, wherein the system is integrated with plurality of processes consisting of array hybridization, image capturing, image analysis, statistical analysis and laboratory information management systems.

14. The computer system according to claim 1, wherein the system is integrated with a plurality of storage means to store the information from the analyzing means.

15. The computer system according to claim 1, wherein the selection means and the presenting means are in graphical user interface (GUI).

16. The computer system according to claim 1, wherein the system is run on a standalone computer.

17. The computer system according to claim 1, wherein the system in run on one or more networked computers.

18. A computer system for analyzing expression information of array hybridized biological samples, the system comprising:
reading modules to read expression information of one or more biological samples from a plurality of data sources;
analyzing modules to analyze the expression information of the one or more samples, wherein the analysis is performed by estimating stastistical variation;
selection modules to select for the one or more analyzed samples by estimating variation from the analyzing modules; and
presenting modules to cause the system to present the expression information of the one or more selected samples.

19. The computer system according to claim 18, wherein the expression information is obtained from biological samples having no external treatment (X₀) and biological samples treated by one or more external factors at plurality of levels including high (Xₜ) and low (Yₜ) levels corresponding to time (t), and wherein the variation is estimated by analyzing means using a Chi Square analysis.

20. The computer system according to claim 19, wherein a Chi Square value is calculated using standard deviation (SD) according to the relation
Chi Square=(Xₜ−X₀)²/SD².

21. The computer system according to claim 19, wherein a Chi Square value is calculated using standard deviation (SD) according to the relation
Chi Square=(Yₜ−X₀)²/SD².

22. The computer system according to claim 19, wherein a Chi Square value is calculated using standard deviation (SD) according to the relation
Chi Square=(Xₜ−Yₜ)²/SD².

23. The computer system according to claim 18, wherein the selection modules further enable the user to select the expression of biological samples showing a probability value of false positive equivalent to 1/(total number of biological samples).

24. The computer system according to claim 18, wherein the variation estimated by the analyzing modules is due to influence of one or more biological samples.

25. The computer system according to claim 18, wherein the variation estimated by analyzing modules is due to one or more external factors influencing the biological samples.

26. The computer system according to claim 18, wherein the variation estimated by the analyzing modules is due to one or more error sources in the process of producing and measuring hybridized arrays.

27. The computer system according to claim 18, wherein the biological samples are a plurality of polynucleotides.

28. The computer system according to claim 18, wherein the biological samples are a plurality of polypeptides.

29. The computer system according to claim 18, wherein the external factor is a drug.

30. The computer system according to claim 18, wherein the system is integrated with a plurality of processes further comprising array hybridization, image capturing, image analysis, statistical analysis and laboratory information management systems.

31. The computer system according to claim 18, wherein the system is integrated with a plurality of storage modules to store the information from the analyzing modules.

32. The computer system according to claim 18, wherein the selection modules and presenting modules are graphical user interfaces (GUI).

33. The computer system according to claim 18, wherein the system is run on a standalone computer.

34. The computer system according to claim 18, wherein the system in run on one or more networked computers.

35. A method for analyzing expression information of biological samples, the method comprising:
reading expression information of one or more samples from plurality of data sources;
analyzing the expression information of the samples, wherein the analysis is performed by estimating statistical variation;
selecting one or more of the analyzed samples by utilizing estimated variation from the analysis; and
presenting expression information of the selected samples.

36. The method according to claim 35, wherein the expression information is obtained from biological samples having no external treatment (X₀) and biological samples treated by one or more external factors at plurality of levels including high (Xₜ) and low (Yₜ) levels corresponding to time (t), and wherein the variation is estimated by analyzing means using a Chi Square analysis.

37. The method according to claim 36, wherein a Chi Square value is calculated using standard deviation (SD) according to the relation
Chi Square=(Xₜ−X₀)²/SD².

38. The method according to claim 36, wherein a Chi Square value is calculated using standard deviation (SD) according to the relation
Chi Square=(Yₜ−X₀)²/SD².

39. The method according to claim 36, wherein a Chi Square value is calculated using standard deviation (SD) according to the relation
Chi Square=(Xₜ−Yₜ)²/SD ².

40. The method according to claim 35, wherein the step of selecting further selects the expression of biological samples showing a probability value of false positive equivalent to
1/(total number of biological samples).

41. The method according to claim 35, wherein the variation estimated by the analyzing means is due to influence of one or more biological samples.

42. The method according to claim 35, wherein the variation estimated by the analyzing means is due to one or more external factors influencing biological samples.

43. The method according to claim 35, wherein the variation estimated by the analyzing means is due to one or more error sources in the process of producing and measuring the array hybridized biological samples.

44. The method according to claim 35, wherein the biological samples are a plurality of polynucleotides.

45. The method according to claim 35, wherein the biological samples are a plurality of polypeptides.

46. The method according to claim 42, wherein the external factor is a drug.

47. A system comprising electronic storage medium having computer readable code contained therein, the electronic storage medium further comprising:
computer readable code that instructs a processor to read expression information of one or more biological samples from a plurality of data sources;
computer readable code that instructs a processor to analyze the expression information of the one or more samples, wherein the analysis is performed by estimating stasistical variation;
computer readable code that instructs a processor to select one or more of the analyzed samples by utilizing the estimated stasistical variation from analyzing means; and
computer readable code that instructs a processor to present the expression information of the biological samples to a user, whereby the computer readable code allows a user to analyze expression information of biological samples.

48. The electronic storage medium according to claim 47, wherein the expression information are obtained from biological samples with no external treatment (X₀) and biological samples treated by one or more external factors at plurality of levels including high (Xₜ) and low (Yₜ) levels corresponding to time (t), and wherein the variation is estimated by analyzing means using a Chi Square analysis.

49. The electronic storage medium according to claim 48, wherein a Chi Square value is calculated using standard deviation (SD) according to the relation Chi Square=(Xₜ−X₀)²/SD²

50. The electronic storage medium according to claim 48, wherein a Chi Square value is calculated using standard deviation (SD) according to the relation Chi Square=(Yₜ−X₀)²/SD².

51. The electronic storage medium according to claim 48, wherein a Chi Square value is calculated using standard deviation (SD) according to the relation Chi Square=(Xₜ−Yₜ)²/SD².

52. The electronic storage medium according to claim 47, wherein the medium further comprises computer readable code that instructs a processor to enable a user to select the expression of biological samples showing a probability value of false positive equivalent to 1/(total number of biological samples).

53. A computer system according to claim 1, wherein reading means are capable of reading physically measured information from plurality of arrays having plurality of replications.

54. A computer system according to claim 1, wherein the biological samples are hybridized to their reverse complement sequences or fragments thereof, and the hybridization is carried out on a glass substrate.
